(19) United States
(12) Patent Application Publication
(10) Pub. No.: US 2024/0300930 A1
(43) Pub. Date: Sep. 12, 2024

(54) NOVEL PROCESS FOR THE PREPARATION OF 2,5-DICHLORO-3-(5-(3,4-DIHYDROXY-5-NITROPHENYL)-1,2,4-OXADIAZOL-3-YL)-4,6-DIMETHYLPYRIDINE-1-OXIDE

(71) Applicant: MSN LABORATORIES PRIVATE LIMITED, R&D CENTER, Hyderabad, Telangana (IN)

(72) Inventors: Thirumalai Rajan SRINIVASAN, Hyderabad, Telangana (IN); Eswaraiah SAJJA, Hyderabad, Telangana (IN); Rajeshwar Reddy SAGYAM, Hyderabad, Telangana (IN); Navin Kumar Reddy KESHAVAREDDY, Hyderabad, Telangana (IN); Ananthan BAKTHAVACHALAM, Hyderabad, Telangana (IN)

(73) Assignee: MSN LABORATORIES PRIVATE LIMITED, R&D CENTER, Hyderabad, Telangana (IN)

(21) Appl. No.: 18/548,119

(22) PCT Filed: Feb. 28, 2022
(86) PCT No.: PCT/IN2022/050170

§ 371 Date: Aug. 28, 2023

(30) Foreign Application Priority Data
Feb. 26, 2021 (IN) 202141008206

(51) Int. Cl. C07D 413/04; A61K 31/4439

(52) U.S. Cl. CPC C07D 413/04; A61K 31/4439

(57) ABSTRACT

The present invention relates to a novel process for the preparation of 2,5-dichloro-3-(5-(3,4-dihydroxy-5-nitrophenyl)-1,2,4-oxadiazol-3-yl)-4,6-dimethylpyridine-1-oxide of formula (I)

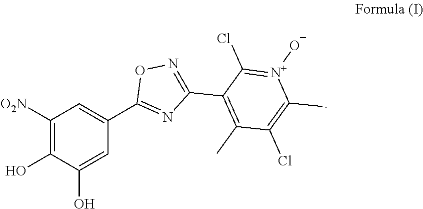 [SMILES: Cc1c(Cl)c(C)[n+]([O-])c(Cl)c1-c1noc(-c2cc(O)c(O)c([N+](=O)[O-])c2)n1]

The present invention also relates to the novel intermediate compounds of formula (V), formula (VII), and their processes for the preparation thereof.

The present invention also relates to the use of compounds of formula (V), formula (VII) intermediate in the preparation of 2,5-dichloro-3-(5-(3,4-dihydroxy-5-nitrophenyl)-1, 2, 4-oxadiazol-3-yl)-4, 6-dimethylpyridine-1-oxide of formula (I).

### RELATED APPLICATION

This application claims the benefit of priority of our Indian patent application number 202141008206 filed on Feb. 26, 2021 which is incorporated herein by reference.

### FIELD OF THE INVENTION

The present invention relates to a novel process for the preparation of 2,5-dichloro-3-(5-(3,4-dihydroxy-5-nitrophenyl)-1,2,4-oxadiazol-3-yl)-4,6-dimethylpyridine-1-oxide formula (I).

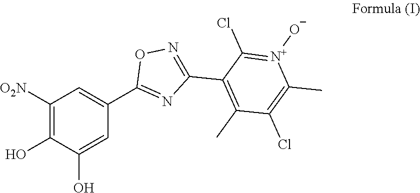 [SMILES: Cc1c(Cl)c(C)[n+]([O-])c(Cl)c1-c1noc(-c2cc(O)c(O)c([N+](=O)[O-])c2)n1]

### BACKGROUND OF THE INVENTION

The compound of formula (I) commonly known as “Opicapone” and it is a catechol-O-methyltransferase (COMT) inhibitor indicated as adjunctive treatment to levodopa/carbidopa in patients with Parkinson's disease (PD) experiencing “off” episodes, and it is approved by USFDA under brand name of ONGENTYS®.

U.S. Pat. No. 8,168,793 B2 (hereinafter referred as US' 793) discloses Opicapone and its process for its preparation thereof. The process disclosed in US '793 is schematically shown below:

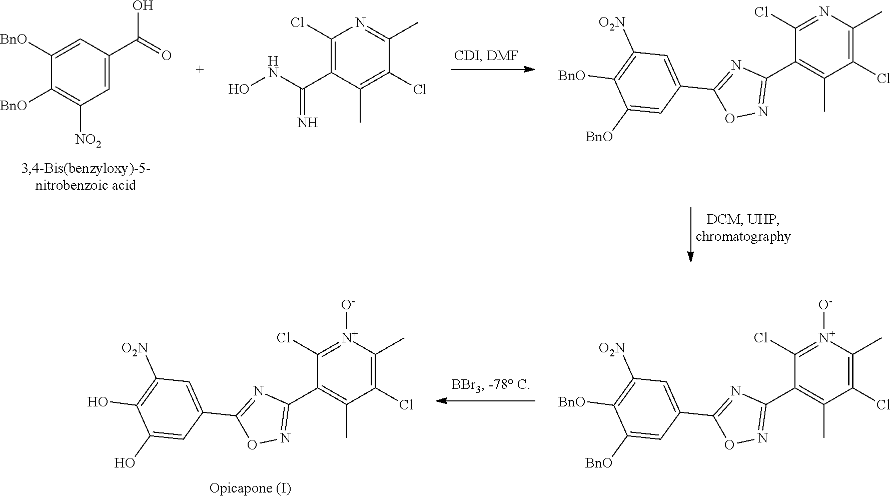 [SMILES: O=C(O)c1cc(OCc2ccccc2)c(OCc2ccccc2)c([N+](=O)[O-])c1 ; Cc1nc(Cl)c(C(=N)NO)c(C)c1Cl ; Cc1nc(Cl)c(-c2noc(-c3cc(OCc4ccccc4)c(OCc4ccccc4)c([N+](=O)[O-])c3)n2)c(C)c1Cl ; Cc1c(Cl)c(C)[n+]([O-])c(Cl)c1-c1noc(-c2cc(O)c(O)c([N+](=O)[O-])c2)n1 ; Cc1c(Cl)c(C)[n+]([O-])c(Cl)c1-c1noc(-c2cc(OCc3ccccc3)c(OCc3ccccc3)c([N+](=O)[O-])c2)n1]

The Opicapone process reported in US '793 is suffered with more number of steps required for the preparation of 3,4-bis(benzyloxy)-5-nitrobenzoic acid. Journal of Medicinal Chemistry 2010, 53, 8, 3396-3411 reported a process for the preparation of 3,4-bis(benzyloxy)-5-nitrobenzoic acid from vanillic acid in three stages. Further, the process reported in US '793 also involves the use of BBr₃ that is hazardous and it is carried out at −78° C. making the above reaction not safe and cumbersome on large scale. The above reported process further involves chromatographic purification which uses ethyl acetate and petroleum ether as eluents which makes the process tedious at commercial scale-up. Therefore, it is necessary to design safe, cost effective, environmentally friendly and easy-to-operate synthetic process with lesser reaction cycle time.

U.S. Pat. No. 9,126,988 B2 (hereinafter referred as US' 988) discloses a process for the preparation of Opicapone. The process disclosed in US' 988 is schematically shown as below:

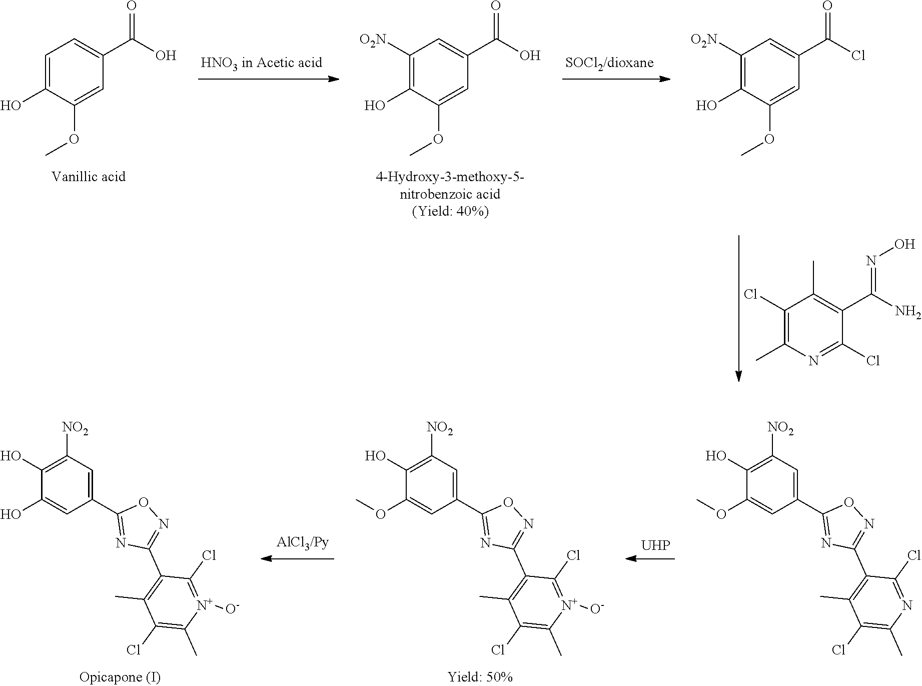

Main drawback of process reported in US '988 is very low yield (i.e. 40%) of 4-hydroxy-3-methoxy-5-nitrobenzoic acid which is prepared from vanillic acid which in turn influences over all yield of Opicapone. Therefore, said process is economically not viable at industrial scale-up due to low yield of 4-hydroxy-3-methoxy-5-nitrobenzoic acid in the beginning stage of the reaction.

In view of the above concerns, there is a need to develop a commercially viable, inexpensive process for the preparation of Opicapone. Inventors of present invention developed a novel process for the preparation of Opicapone.

### SUMMARY OF INVENTION

In first embodiment, the present invention provides a novel process for preparation of compound of formula (V).

In second embodiment, the present invention provides a novel process for preparation of compound of formula (IV).

In third and fourth embodiments, the present invention provide a novel process for preparation of Opicapone.

In fifth embodiment, the present invention provides a novel intermediate compounds of formulae V and VII.

In sixth embodiment, the present invention provides a process for purification of Opicapone.

In seventh embodiment, the present invention provides particle size distribution of Opicapone.

### BRIEF DESCRIPTION OF THE DRAWINGS

FIG. 1: Illustrates the PXRD pattern of 4-(3-(2,5-dichloro-4,6-dimethylpyridin-3-yl)-1,2,4-oxadiazol-5-yl)-2-methoxyphenol of formula-Va obtained according example-11.

FIG. 2: Illustrates the PXRD pattern of Opicapone obtained according example-15.

### DETAILED DESCRIPTION OF INVENTION

The term “solvent” used in the present invention refers to “hydrocarbon solvents” such as n-hexane, n-heptane, cyclohexane, benzene, toluene, pentane, cycloheptane, ethyl benzene, m-, o-, or p-xylene or and the like; “ether solvents” such as dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, methyl tert-butyl ether, 1,2-dimethoxy ethane and the like; “ester solvents” such as methyl acetate, ethyl acetate, isopropyl acetate, n-butyl acetate and the like; “polar-aprotic solvents” such as dimethylacetamide (DMA), dimethylformamide (DMF), dimethylsulfoxide (DMSO), N-methylpyrrolidone (NMP) and the like; “chloro solvents” such as methylene chloride, dichloroethane, chloroform, carbon tetrachloride, chlorobenzene and the like; “ketone solvents” such as acetone, methyl ethyl ketone, pentanone, methyl isobutylketone and the like; “nitrile solvents” such as acetonitrile, propionitrile, isobutyronitrile and the like; “alcohol solvents” such as methanol, ethanol, n-propanol, isopropanol, n-butanol, isobutanol, t-butanol, n-pentanol, isopentanol, 2-nitroethanol, ethylene glycol, 2-methoxyethanol, 1, 2-ethoxyethanol, diethylene glycol, 1, 2, or 3-pentanol, neo-pentyl alcohol, t-pentyl alcohol, diethylene glycol monoethyl ether, benzyl alcohol, phenol, or glycerol and the like; “polar solvents” such as water or mixtures thereof.

As used herein the present invention the term “halogen” refers to fluorine, chlorine, bromine and Iodine.

As used herein the present invention the term “alkylsulfonyl” refers methanesulfonyloxy, ethanesulfonyloxy and same like.

As used herein the present invention the term “arylsulfonyl” refers p-toulenesulfonyloxy, benzenesulfonyloxy and same like.

As used herein the present invention the term “alkoxy” refers methoxy, ethoxy, propoxy, i-propoxy, i-butoxy, n-butoxy and same like.

In first embodiment, the present invention provides a novel process for preparation of compound of formula (V), comprising:
a) reacting compound of formula (III) with compound of formula (IV) to provide the compound of formula (V)

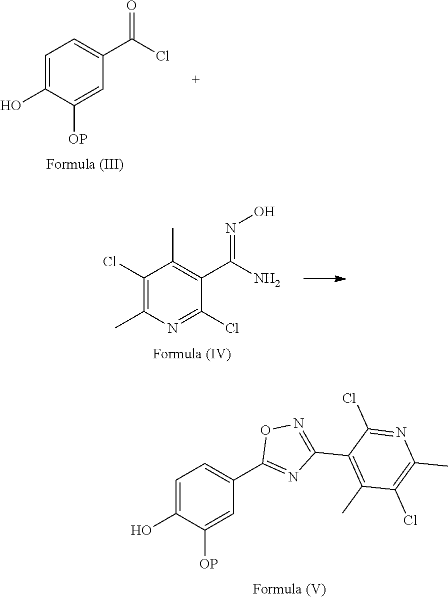

wherein, P refers to a protecting group such as methyl, ethyl, n-propyl, isopropyl, n-butyl, isobutyl, acetyl, trifluoro acetyl, benzyl, benzoyl and the like thereof
b) converting compound of formula (V) to Opicapone of formula (I).

In an aspect of the first embodiment the reaction of step-a) is carried out in presence of a base and solvent. The base is selected from organic base such as triethyl amine, methyl amine, ethyl amine, diisopropylethylamine, 2,6-lutidine and pyridine. The solvent is selected from polar-aprotic solvents such as dimethylacetamide (DMA), dimethylformamide (DMF), dimethylsulfoxide (DMSO), N-methylpyrrolidone (NMP) or mixtures thereof, “hydrocarbon solvents” such as n-hexane, n-heptane, cyclohexane, benzene, toluene, pentane, cycloheptane, ethyl benzene, m-, o-, or p-xylene or mixtures thereof; ketone solvents” such as acetone, methyl ethyl ketone, pentanone, methyl isobutyl ketone or mixtures thereof, “nitrile solvents” such as acetonitrile, propionitrile, isobutyronitrile or mixtures thereof; ether solvents” such as dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, methyl tert-butyl ether, 1,2-dimethoxy ethane or mixtures thereof, “ester solvents” such as methyl acetate, ethyl acetate, isopropyl acetate, n-butyl acetate or mixtures thereof.

In second embodiment, the present invention provides a novel process for preparation of compound of formula (VI), comprising:
a) nitrating the compound of formula (V) to provide compound of formula (VI)

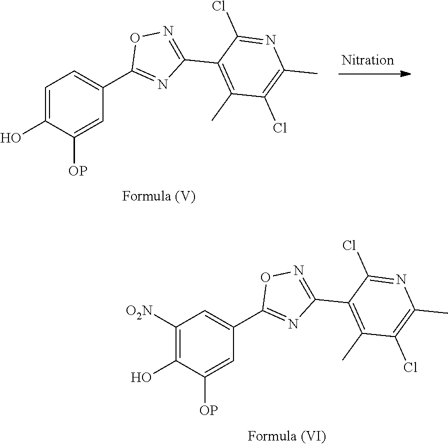

b) converting compound of formula (VI) to Opicapone of formula (I).

In the first aspect of the second embodiment, nitration of step-a) is carried out using nitrating mixture (a mixture of nitric acid and sulfuric acid or a mixture of nitric acid and acetic acid), sodium nitrate, potassium nitrate, calcium nitrate, cupric nitrate and the like or mixtures thereof.

In the second aspect of second embodiment, compound of formula (V) can be prepared according the first embodiment.

In third embodiment, the present invention provides a novel process for preparation of Opicapone of formula (I), comprising:
a) reacting compound of formula (III) with compound of formula (IV) to provide compound of formula (V)

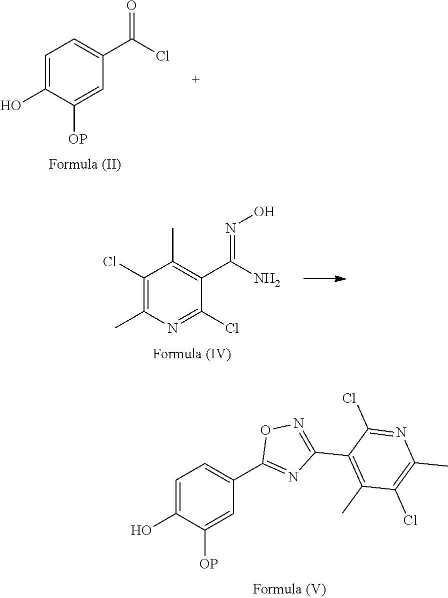

wherein, P refers to a protecting group such as methyl, ethyl, n-propyl, isopropyl, n-butyl, isobutyl, acetyl, trifluoro acetyl, benzyl, benzoyl and the like thereof
b) converting compound of formula (VI) to Opicapone of formula (I).
   wherein the reaction conditions of step-a) is same as described in first and second embodiments.

In the fourth embodiment, the process for the preparation of Opicapone of formula (I) comprises:
a) reacting the compound of formula (II) with compound of formula (IV) to provide the compound of formula (VII),

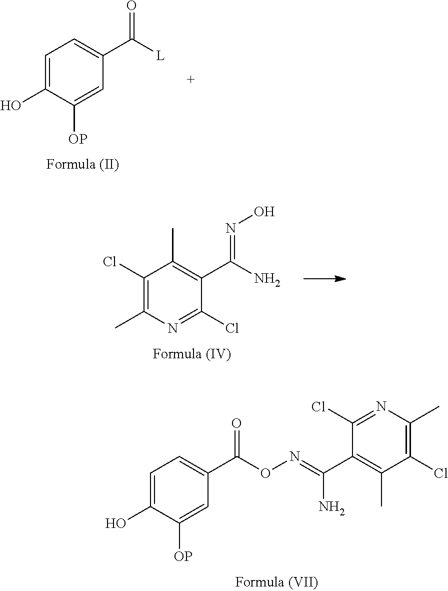

wherein, P refers to a protecting group such as methyl, ethyl, n-propyl, isopropyl, n-butyl, isobutyl, acetyl, trifluoro acetyl, benzyl, benzoyl and the like thereof;
L is —OH or any leaving group selected from halo, alkoxy, alkylsulofonyl or aryl sulfonyl;
b) cyclizing the compound of formula (VII) to provide compound of formula (V),

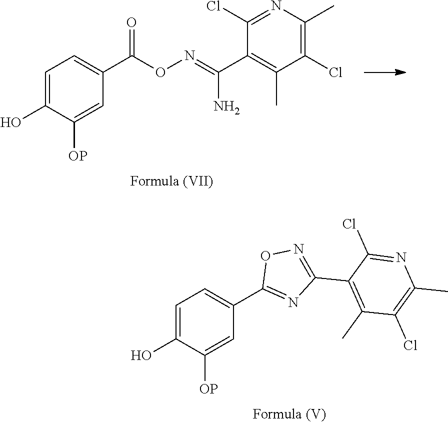

wherein, P refers to a protecting group such as methyl, ethyl, n-propyl, isopropyl, n-butyl, isobutyl, acetyl, trifluoro acetyl, benzyl, benzoyl and the like thereof,
c) nitrating the compound of formula (V) to provide compound of formula (VI)

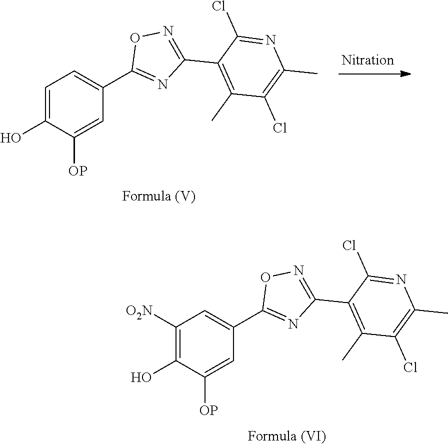

d) converting compound of formula (VI) to Opicapone of formula (I).

First aspect of fourth embodiment, the reaction in step-a) and b) is carried out in presence of base is refers to inorganic base or organic base; inorganic base selected from “alkali metal carbonates” such as sodium carbonate, potassium carbonate, lithium carbonate and the like; “alkali metal bicarbonates” such as sodium bicarbonate, potassium bicarbonate and the like; “alkali metal hydroxides” such as sodium hydroxide, potassium hydroxide, lithium hydroxide and the like; alkali metal hydrides such as sodium hydride, potassium hydride, lithium hydride and the like; alkali metal amides such as sodium amide, potassium amide, lithium amide and the like; ammonia such as liquor ammonia, ammonia gas, alcoholic ammonia and the like; and organic base selected from dimethylamine, diethylamine, diisopropyl amine, diisopropylethylamine, diisobutylamine, triethylamine, tertiary butyl amine, benzyl amine, “alkali metal alkoxides” such as sodium methoxide, sodium ethoxide, potassium methoxide, potassium ethoxide, sodium tert.butoxide, potassium tert.butoxide, lithium tert.butoxide and the like; pyridine, 4-dimethylaminopyridine (DMAP), N-methyl morpholine (NMM), 2,6-lutidine, lithium diisopropylamide; organosilicon bases such as lithium hexamethyldisilazide (LiHMDS), sodium hexamethyldisilazide (NaHMDS), potassium hexamethyldisilazide (KHMDS) or mixtures thereof; wherein base is refers to inorganic base or organic base; reaction conditions of step-c) is same as described in second and third embodiments.

Second aspect of fourth embodiment, the reaction in step-a) to step-c) are carried out in a solvent selected from “hydrocarbon solvents” such as n-pentane, n-hexane, n-heptane, cyclohexane, petroleum ether, benzene, toluene, xylene and mixtures thereof, “ether solvents” such as dimethyl ether, diethyl ether, diisopropyl ether, methyl tert-butyl ether, 1,2-dimethoxyethane, tetrahydrofuran, 1,4-dioxane and mixtures thereof; “ester solvents” such as methyl acetate, ethyl acetate, n-propyl acetate, isopropyl acetate, n-butyl acetate, isobutyl acetate, tert-butyl acetate and mixtures thereof, “polar-aprotic solvents” such as dimethylacetamide, dimethylformamide, dimethylsulfoxide, N-methylpyrrolidone (NMP) and mixtures thereof; “chloro solvents” such as dichloromethane, dichloroethane, chloroform, carbon tetrachloride and mixtures thereof, “ketone solvents” such as acetone, methyl ethyl ketone, methyl isobutyl ketone and mixtures thereof, “nitrile solvents” such as acetonitrile, propionitrile, isobutyronitrile and mixtures thereof; “alcohol solvents” such as methanol, ethanol, n-propanol, isopropanol (or) isopropyl alcohol, n-butanol, iso-butanol, 2-butanol, tert-butanol, ethane-1,2-diol, propane-1,2-diol and mixtures thereof, “polar solvents” such as water, formic acid, acetic acid and the like or mixture of any of the afore mentioned solvents.

Third aspect of the fourth embodiment, the compound of formula (VII) is isolated as a solid.

Fourth aspect of the fourth embodiment, the compound of formula (V) is prepared without isolation of compound of formula (VII) as a solid.

Conversion of the compound of formula-(VI) to Opicapone of formula (I) can be done by the method described in the instant application or the process described in US 2018/0370958 A1 or any other process known in the art.

Compound of formula (II) is prepared by the known methods in the literature.

In fifth embodiment, the present invention provides novel compounds of formulae (VII) and (V).

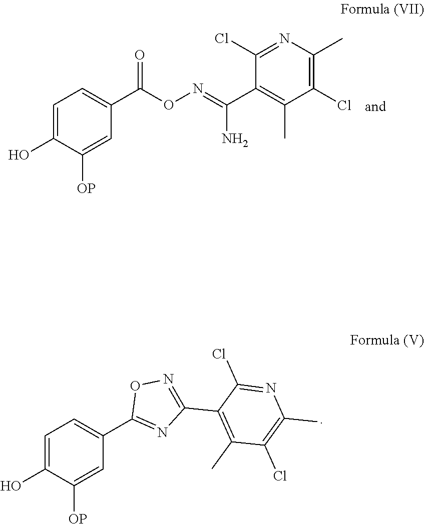

wherein, P refers to a protecting group such as methyl, ethyl, n-propyl, isopropyl, n-butyl, isobutyl, acetyl, trifluoro acetyl, benzyl, benzoyl and the like thereof.

In first aspect of fifth embodiment, novel compounds of formulae (VII) and (V) are used in the preparation of Opicapone of formula (I).

In second aspect of fifth embodiment, the present invention provides novel compound of formulae (VIIa) and (Va).

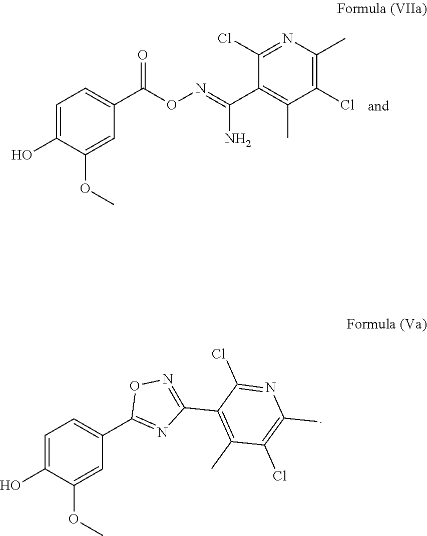 [SMILES: COc1cc(C(=O)O/N=C(\N)c2c(Cl)nc(C)c(Cl)c2C)ccc1O ; COc1cc(-c2nc(-c3c(Cl)nc(C)c(Cl)c3C)no2)ccc1O]

In third aspect of fifth embodiment, novel compounds of formulae (VIIa) and formula (Va) are used in the preparation of Opicapone of formula (I).

In the sixth embodiment of present invention provides process for the purification of Opicapone of formula (I) comprises:
a) dissolving the Opicapone of formula (I) in the mixture of dimethyl sulfoxide and ethyl acetate,
b) combining an alcohol solvent or water with the solution obtained in step-a) and
c) isolating the Opicapone of formula (I).
   wherein the dissolving of Opicapone of formula (I) in step-a) can be done at temperature ranging from about 25° C. to reflux temperature of solvent or mixture of solvents used; isolating Opicapone in step-c) is by solvent removal by known techniques which are selected from cooling the mixture to lower temperatures to precipitate the solid followed by filtration of the mixture; alcohol solvent in step-b) is selected from methanol, ethanol, n-propanol, iso-propanol, n-butanol, iso-butanol, 2-butanol, tert-butanol, ethane-1,2-diol, propane-1,2-diol and mixtures thereof.

In seventh embodiment, Opicapone of formula (I) having particle size distribution characterized by: (i) a D10 value is less than about 4 μm; (ii) a D50 value is less than about 10 μm; or (iii) a D90 value is less than about 100 μm; or a combination of (i), (ii) and/or (iii).

The skilled artisan knows that the results of PSD determination by one technique can be correlated with results from another technique on an empirical basis by routine experimentation.

More specifically, unless otherwise indicated, the particle size was determined using a Leica polarized light microscope (Model, DM2700P) equipped with 5.0 mega pixel camera (Image ProVsion Technology, model: ISH500) and test sample was taken in to a microscopic glass slide by spreading the powder sample properly on the microsocpic slide. The prepared samples particle size is analyzed using ipvPSA software (version 2.1).

The median particle size (D50), which is also denoted D50-value of the integral volume distribution, is defined in the context of this invention as the particle diameter, at which 50 percent by volume of the particles have a smaller diameter than the diameter which corresponds to the D50-value. Likewise, 50 percent by volume of the particles have a larger diameter than the D50-value. Analogously, the D90-value of the integral volume distribution is defined as the particle diameter, at which 90 percent by volume of the particles have a smaller diameter than the diameter, which corresponds to the D90-value. Correspondingly, the D10-value of the integral volume distribution is defined as the particle diameter, at which 10 percent by volume of the particles have a smaller diameter than the diameter, which corresponds to the D10-value.

Opicapone prepared according to the present invention having a purity of about 95%; preferably greater than about 97%; more preferably greater than about 99% as measured by HPLC.

The following impurities are observed during the synthesis of the Opicapone as per the present invention. Along with these impurities, the starting materials are well controlled as per ICH guide lines in the Opicapone of formula-(I).

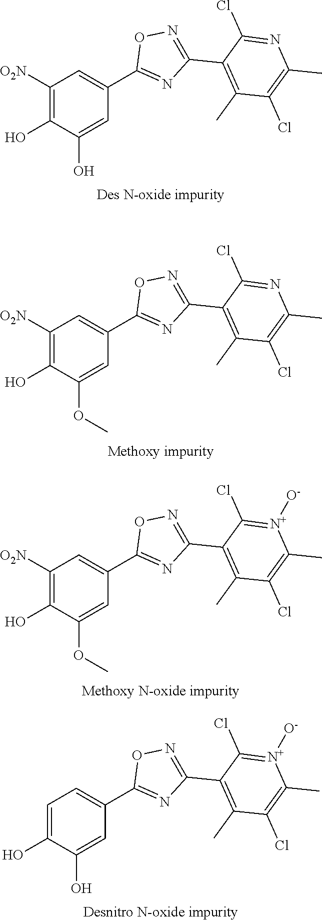 [SMILES: Cc1nc(Cl)c(-c2noc(-c3cc(O)c(O)c([N+](=O)[O-])c3)n2)c(C)c1Cl ; COc1cc(-c2nc(-c3c(Cl)nc(C)c(Cl)c3C)no2)cc([N+](=O)[O-])c1O ; COc1cc(-c2nc(-c3c(C)c(Cl)c(C)[n+]([O-])c3Cl)no2)cc([N+](=O)[O-])c1O ; Cc1c(Cl)c(C)[n+]([O-])c(Cl)c1-c1noc(-c2ccc(O)c(O)c2)n1]

### HPLC Method of Analysis:

Opicapone of formula (I) and its related substances were analyzed by HPLC with the following chromatographic conditions:
Apparatus: A liquid chromatographic system is to be equipped with variable wavelength UV-Detector and integrator.
Column: Kinetex Biphenyl 100 A°, 250×4.6 mm, 5.0 μm (Make: Phenomenex, P/No:00G-4627-E0); Ghost-Buster Column: Ghost buster 50 mm×4.6 mm [Make: Welch & Part No: 06100-31000]; Wavelength: 220 nm; Column temperature: 45° C.; Injection volume: 10 μL; Elution: Gradient; Diluent: *Chilled 0.1% Orthophosphoric acid in Acetonitrile:Water (90:10) v/v (With aid of ice or Refrigerator) Note: * Temperature in between 0° C. and 10° C.
Needle wash: Diluent; Sample Concentration: 0.5 mg/mL;

### Buffer Preparation:

Transfer accurately 2.0 mL of Perchloric acid into a 1000 mL of Milli-Q water into a suitable clean and dry beaker and adjust the pH 2.0±0.1 with 20% KOH Solution
ii) Filter the above solution through 0.22 μm polyvinylidene fluoride membrane filter paper and sonicate about 2 minutes to degas it.
Mobile phase-A: Buffer (100%)
Mobile phase-B Preparation: Transfer accurately about 800 mL of Acetonitrile and 200 mL of Methanol into a 1000 mL mobile phase bottle, mix well and sonicate to degas it. (Acetonitrile: Methanol) (80:20 v/v)
Note: i) Opicapone Sample solution found to be stable up to 5 days at 5° C. temperature and Mobile Phase found to be stable up to 5 Days at Room Temperature based on method validation data
0.1% Orthophosphoric acid in Acetonitrile: Water (90:10) v/v preparation:
Transfer accurately about 90 mL of Acetonitrile and 10 mL of water into a 100 mL volumetric flask add 0.1 mL (100 μL) of Orthophosphoric acid (85%) and mix well.
20% KOH (Potassium hydroxide) solution preparation:
Weigh accurately 20 g of KOH pellets into 100 mL of volumetric flask, add about 50-60 mL of water and sonicate to dissolve. Make up to the mark with diluent and mix well.

In an embodiment of the present invention provides a pharmaceutical composition comprising Opicapone and one or more pharmaceutically acceptable excipient. As used herein, the term “pharmaceutical compositions” or “pharmaceutical formulations” include tablets, pills, powders, liquids, suspensions, emulsions, granules, capsules, suppositories, or injection preparations.

Wherein the “excipient” is selected from but not limited to polyvinylpyrrolidone (povidone or PVP), polyvinylpolypyrrolidone, polysorbate, copovidone, cross linked polyvinyl pyrrolidone (crospovidone), polyethylene glycol (macrogol or PEG), polyvinyl alcohol, polyvinyl chloride, polyvinyl acetate, propylene glycol, cellulose, cellulose acetate phthalate (CAP), methyl cellulose, carboxymethyl cellulose (CMC, its sodium and calcium salts), carboxymethylethyl cellulose (CMEC), ethyl cellulose, hydroxymethyl cellulose, ethyl hydroxyethyl cellulose, hydroxyethylcellulose, hydroxypropyl cellulose (HPC), hydroxypropyl cellulose acetate succinate, hydroxypropyl methyl cellulose (hypromellose or HPMC), hydroxypropyl methylcellulose acetate succinate (HPMC-AS), hydroxyethyl methyl cellulose succinate (HEMCS), hydroxypropyl cellulose acetate succinate (HPCAS), hydroxypropyl methylcellulose phthalate (HPMC-P), hydroxypropyl methylcellulose acetate phthalate, microcrystalline cellulose (MCC), cross linked sodium carboxymethyl cellulose (croscarmellose sodium), cross linked calcium carboxymethyl cellulose, magnesium stearate, aluminium stearate, calcium stearate, magnesium carbonate, talc, iron oxide (red, yellow, black), stearic acid, dextrates, dextrin, dextrose, sucrose, glucose, xylitol, lactitol, sorbitol, mannitol, maltitol, maltose, raffinose, fructose, maltodextrin, anhydrous lactose, lactose monohydrate, starches such as maize starch or corn starch, sodium starch glycolate, sodium carboxymethyl starch, pregelatinized starch, gelatin, sodium dodecyl sulfate, edetate disodium, sodium phosphate, sodium lauryl sulfate, triacetin, sucralose, calcium phosphate, polydextrose, α-, β-, γ-cyclodextrins, sulfobutylether beta-cyclodextrin, sodium stearyl fumarate, fumaric acid, alginic acid, sodium alginate, propylene glycol alginate, citric acid, succinic acid, carbomer, docusate sodium, glyceryl behenate, glyceryl stearate, meglumine, arginine, polyethylene oxide, polyvinyl acetate phthalates and the like.

### PXRD Method of Analysis:

The PXRD analysis of compounds of the present invention is carried out by using BRUKER/D8 ADVANCE X-Ray diffractometer using CuKα radiation of wavelength 1.5406A° and at a continuous scan speed of 0.03°/min.

The process described in the present invention is illustrated in examples below. These examples are provided as illustration only and therefore should not be construed as limitation of the scope of the invention:

### EXAMPLES

### Example-1: Preparation of 2,5-dichloro-N′-hydroxy-4,6-dimethylnicotinimidamide

2,5-Dichloro-4,6-dimethylnicotinonitrile (150 gm) was added to methanol (675 ml), water (1125 ml), 1,10-phenanthroline (2.03 gm) and aqueous hydroxylamine (177 ml) at 25-30° C. and stirred for 10 min at same temperature. Raised the temperature of the reaction mixture to 75-80° C. and stirred for 7 hrs. Cooled the reaction mixture to 10-15° C. and stirred for 1 hr. Filtered the obtained compound, washed with water and then dried to get the title compound (Yield: 130 gm).

### Example-2: Preparation of 4-(3-(2,5-dichloro-4,6-dimethylpyridin-3-yl)-1,2,4-oxadiazol-5-yl)-2-methoxyphenol

Vanillic acid (39.51 gm) was added to toluene (250 ml), thionyl chloride (33.04 gm) and DMF (2.03 gm) at 25-30° C. Raised the temperature of the reaction mixture to 65-70° C. and stirred for 2 hrs. Distilled off the solvent from the reaction mixture to get 4-hydroxy-3-methoxybenzoyl chloride compound and dissolved in toluene (250 ml) to get a solution.

Dimethyl acetamide (300 ml) was added to (Z)-2,5-dichloro-N′-hydroxy-4,6-dimethylnicotinimidamide (50 gm) in a separate flask at 25-30° C. To this reaction mixture, the above obtained 4-hydroxy-3-methoxybenzoyl chloride in toluene was added at 0-5° C. and stirred for 1 hr at same temperature. Pyridine (143.62 gm) was added to the reaction mixture at 0-5° C. and stirred for 2 hrs at 25-30° C. Heated the reaction mixture to 120-125° C. and stirred for about 12 hrs at same temperature. Cooled the reaction mixture to 25-30° C. and washed with water. Separated the organic and aqueous layers and extracted the aqueous layer with toluene. Combined the total organic layers and washed with aqueous HCl solution. Separated the organic and aqueous layers and extracted the organic layer with aqueous NaOH solution. Separated the aqueous and organic layers and washed the aqueous layer with toluene. Separated the aqueous layer and added to toluene followed by pH adjustment between 2-3 with HCl. Separated the organic and aqueous layers and extracted the aqueous layer with toluene. Combined the total organic layers and distilled off the solvent under reduced pressure. Toluene was added to the obtained compound and stirred for 1 hr at the same temperature. Filtered the obtained compound, washed with toluene and then dried to get the title compound (Yield 40 gm).

### Example 3: Preparation of 4-(3-(2,5-dichloro-4,6-dimethylpyridin-3-yl)-1,2,4-oxadiazol-5-yl)-2-methoxy-6-nitrophenol

4-(3-(2,5-Dichloro-4,6-dimethylpyridin-3-yl)-1,2,4-oxadiazol-5-yl)-2-methoxy phenol (50 g) was added to acetic acid (500 ml) and nitric acid (17.21 gm) at 10-15° C. and stirred for about 30 min at same temperature. Raised the temperature of the reaction mixture to 25-30° C. and stirred for about 12 hrs at same temperature. Quenched the obtained reaction mixture with water. Filtered the compound followed by slurried in water for 2 hr. Filtered the obtained material, washed with water and then dried to get the title compound (Yield: 45 gm).

### Example 4: Preparation of 5-(3-(2,5-dichloro-4,6-dimethylpyridin-3-yl)-1,2,4-oxadiazol-5-yl)-3-nitrobenzene-1,2-diol

4-(3-(2,5-Dichloro-4,6-dimethylpyridin-3-yl)-1,2,4-oxadiazol-5-yl)-2-methoxy-6-nitrophenol (55 gm) was dissolved in NMP (495 ml) at 25-30° C. and cooled the reaction mixture to 0-5° C. Aluminium chloride (23.19 gm) and pyridine (44.43 gm) were added to the reaction mixture at 0-5° C. Raised the temperature of the reaction mixture to 55-60° C. and stirred for about 6 hrs at same temperature. Cooled the reaction mixture to 25-30° C. and added aqueous hydrochloric acid solution and stirred for about 1 hr. Filtered the obtained compound, washed with water and then dried to get the title compound (Yield: 47 gm).

### Example 5: Preparation of 2,5-Dichloro-3-(5-(3,4-dihydroxy-5-nitrophenyl)-1,2,4-oxadiazol-3-yl)-4,6-dimethylpyridine 1-oxide (Opicapone)

5-(3-(2,5-Dichloro-4,6-dimethylpyridin-3-yl)-1,2,4-oxadiazol-5-yl)-3-nitrobenzene-1,2-diol (10 gm) was added to methylene chloride (100 ml) at 25-30° C. and cooled the reaction mixture to 0-5° C. Urea hydrogen peroxide (23.69 gm) and trifluoroacetic anhydride (74.03 gm) were added to the reaction mixture. Raised the temperature of the reaction mixture to 25-30° C. and stirred for about 16 hrs at same temperature. Washed the reaction mixture with water and separated the organic and aqueous layers. Extracted the aqueous layer with methylene chloride. Combined the total organic layers and distilled off the solvent completely from the organic layer. Methanol (100 ml) was added to the obtained compound at 65-70° C. and stirred for 1 hr at same temperature. Filtered the obtained compound, washed with methanol and then dried to get the title compound (Yield: 8.5 gm).

### Example-6: Purification of 2,5-dichloro-3-(5-(3,4-dihydroxy-5-nitrophenyl)-1,2,4-oxadiazol-3-yl)-4,6-dimethylpyridine 1-oxide (Opicapone)

2,5-Dichloro-3-(5-(3,4-dihydroxy-5-nitrophenyl)-1,2,4-oxadiazol-3-yl)-4,6-dimethylpyridine-1-oxide (5 gm) was dissolved in DMF (50 ml) at 55-60° C. Filtered the solution through hyflow bed. Methanol (100 ml) was added to the obtained filtrate at 55-60° C. and stirred 1 hr at same temperature. Cooled the reaction mixture to 25-30° C. and stirred for about 1 hr at same temperature. Filtered the obtained compound, washed with methanol and then dried to get the title compound (Yield: 3 g).

### Example-7: Preparation of 4,6-dimethyl-2-oxo-1,2-dihydropyridine-3-carbonitrile

Aqueous potassium carbonate solution (potassium carbonate 13.80 g in 150 ml of water) was added to the mixture of acetylacetone (50 g) and cyanoacetamide (41.99 g) at 25-30° C., heated the reaction mixture to 55-60° C. and stirred at the same temperature. Cooled the reaction mixture to 25-30° C. and acidified using aqueous hydrochloric acid solution. Cooled the reaction mixture to 0-5° C. and stirred at the same temperature. Filtered the solid, washed with water and dried to get the title compound. (Yield: 70 g)

### Example-8: Preparation of 5-chloro-4,6-dimethyl-2-oxo-1,2-dihydropyridine-3-carbonitrile

Sulfuryl chloride (81.87 g) was slowly added to the pre-cooled mixture of 4,6-dimethyl-2-oxo-1,2-dihydropyridine-3-carbonitrile (50 g) and acetonitrile (250 ml) at 0-5° C., raised the temperature to 25-30° C. and stirred at the same temperature. Water added to the reaction mixture, neutralized with aqueous potassium carbonate solution and stirred at the same temperature. Filtered the solid. Slurried the obtained solid in the mixture of water and acetonitrile at 25-30° C. and dried to get the title compound. (Yield: 45 g).

### Example-9: Preparation of 2,5-Dichloro-4,6-dimethylnicotinonitrile

Tetramethyl ammonium chloride (15.0 g) was added to the mixture of 5-chloro-4,6-dimethyl-2-oxo-1,2-dihydropyridine-3-carbonitrile (50.0 g) and acetonitrile (150 ml) at 25-35° C. and stirred at the same temperature. Distilled off solvent and co-distilled with acetonitrile. Acetonitrile (150 ml) added to obtained compound and cooled to 25-35° C. Dimethyl formamide (20.01 g) was added to the reaction mixture at 25-30° C. and stirred at the same temperature. Cooled the reaction mixture to 0-5° C. and phosphoryl chloride (100 ml) was slowly added to it. Heated the reaction mixture to 85-90° C. and stirred at the same temperature. Slowly quenched the above reaction mixture into pre cooled water at 10-15° C. Filtered the obtained compound and washed with water. Obtained compound added to isopropyl alcohol (150 ml) at 25-30° C., heated the reaction mixture to 85-90° C. and stirred at the same temperature. Cooled the reaction mass to 25 to 30° C. and at the same temperature. Filtered solid, washed with isopropyl alcohol and dried to get obtained compound.

Yield: (40 g)

### Example-10: Preparation of 2,5-dichloro-N′-hydroxy-4,6-dimethylnicotinimidamide of Formula-IV

Lithium hydroxide monohydrate (43.09 g) was added to mixture of hydroxylamine hydrochloride (129.61 g) and water (450 ml) at 25-30° C. Aqueous ammonia solution (150 ml) and water (150 ml) were added. Heated the reaction mixture to 50-55° C. and stirred at the same temperature. The above obtained solution was slowly added to the mixture of 2,5-dichloro-4,6-dimethylnicotinonitrile (75 gm), methanol (450 ml), and 1,10-phenanthroline (1.01 gm). Heated the reaction mixture to 75-80° C. and stirred for 6 hrs. Cooled the reaction mixture to 5-10° C. and stirred for 1 hour. Filtered the obtained compound and washed with water. Water (600 ml) was added to obtained solid at 25-30° C., cooled to 0-10° C. and stirred at the same temperature. Filtered the obtained solid, washed with water and dried to get the title compound. Yield: 65.5 gm.

### Example-11: Preparation of 4-(3-(2,5-dichloro-4,6-dimethylpyridin-3-yl)-1,2,4-oxadiazol-5-yl)-2-methoxyphenol of Formula-Va (P=Me in Formula-V)

Thionyl chloride (38.12 gm) was slowly added to the pre-cooled mixture of vanillic acid (46.69 gm), dichloromethane (250 ml) and DMF (15.61 gm) at 0-5° C. and stirred at the same temperature. Distilled off the solvent from the reaction mixture to get 4-hydroxy-3-methoxybenzoyl chloride. Dimethyl acetamide (250 ml) was added to the obtained compound at 0-5° C. A solution of 2,5-dichloro-N′-hydroxy-4,6-dimethylnicotinimidamide (50 gm) in dimethyl acetamide (300 ml) was added to above solution at 0-5° C. and stirred at same temperature. 2,6-Lutidine (45.77 gm) was slowly added to the reaction mixture at 0-5° C. and stirred at the same temperature. Water was added to the reaction mixture at 0-5° C. The obtained reaction mixture was slowly quenched into water and stirred at the same temperature. Filter the solid and washed with water to get 2,5-dichloro-N′-((4-hydroxy-3-methoxybenzoyl)oxy)-4,6-dimethylnicotinimidamide. Obtained solid was added to solution of potassium carbonate (38.35 gm) in water (750 ml), heated the reaction mixture to 90-95° C. and stirred at the same temperature. Cooled the reaction mixture to 25-30° C. and stirred at the same temperature. Filtered the solid and washed with water. The obtained solid was added to the mixture of water (400 ml), methanol (400 ml) and potassium carbonate (38.4 gm) at 25-30° C., heated the mixture to 75-80° C. and stirred at the same temperature. Cooled the reaction mixture to 25-30° C. and acidified with acetic acid and stirred at the same temperature. Filtered the obtained solid, washed with water and dried to get the title compound (Yield 60.50 gm).

### Example 12: Preparation of 4-(3-(2,5-dichloro-4,6-dimethylpyridin-3-yl)-1,2,4-oxadiazol-5-yl)-2-methoxy-6-nitrophenol of Formula-VIa (P=Me in Formula-VI)

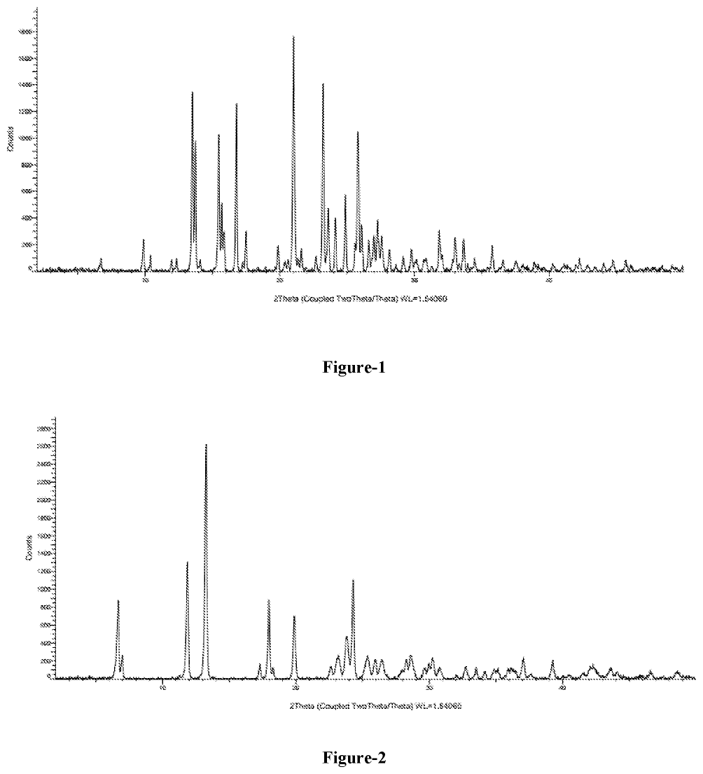

Nitric acid (18.88 gm) was slowly added to pre-cooled mixture of 4-(3-(2,5-Dichloro-4,6-dimethylpyridin-3-yl)-1,2,4-oxadiazol-5-yl)-2-methoxy phenol (50 g), acetic acid (50 ml) and dichloromethane (400 ml) at 0-5° C. and stirred for about 2 hours at same temperature. Reaction mixture was quenched with water, separated the both organic and aqueous layers and aqueous layer was extracted with dichloromethane. Combined the organic layers and washed with water. Distilled off solvent from organic layer and co-distilled with methanol. Recrystallized the obtained compound in methanol to get the title compound (Yield: 45.2 gm). PXRD pattern of the obtained compound is illustrated in FIG. 1.

### Example 13: Preparation of 5-(3-(2,5-dichloro-4,6-dimethylpyridin-3-yl)-1,2,4-oxadiazol-5-yl)-3-nitrobenzene-1,2-diol

Aluminium chloride (21 gm) was added in lot-wise to the mixture of 4-(3-(2,5-Dichloro-4,6-dimethylpyridin-3-yl)-1,2,4-oxadiazol-5-yl)-2-methoxy-6-nitrophenol (50 gm) and N-methyl pyrrolidine (250 ml) at 25-30° C. and stirred at the same temperature. Heated the reaction mixture to 55-60° C. and stirred for about 2 hours at same temperature. Cooled the reaction mixture to 25-30° C. and quenched the reaction mixture into aqueous hydrochloric acid solution. Filtered the obtained compound, washed with water and then dried to get the title compound (Yield: 46 gm).

### Example 14: Preparation of 2,5-Dichloro-3-(5-(3,4-dihydroxy-5-nitrophenyl)-1,2,4-oxadiazol-3-yl)-4,6-dimethylpyridine 1-oxide (Opicapone)

Urea hydrogen peroxide (37.32 gm) lot wise slowly added to the mixture of 5-(3-(2,5-Dichloro-4,6-dimethylpyridin-3-yl)-1,2,4-oxadiazol-5-yl)-3-nitrobenzene-1,2-diol (45 gm), trifluoroacetic anhydride (115.5 gm) and methylene chloride (500 ml) at 25-35° C. and stirred at the same temperature. Aqueous sodium sulfite solution was added to the reaction mixture at 25-30° C. and stirred at the same temperature. Filtered the solid, washed with water and dried to get the title compound. Dissolved the above obtained Opicapone in the mixture of dimethyl sulfoxide (135 ml) and ethyl acetate (315 ml) at 60-65° C. and methanol (225 ml) was added to the obtained solution and stirred at the same temperature for 60 minutes. Methanol (675 ml) was added to reaction mass at 60-65° C. and stirred for 1 hour at the same temperature. Cooled the reaction mass to 25-30° C. and stirred for 60 minutes at the same temperature. Filter the solid, washed with mixture for methanol and water to get the title compound. Repeated same purification once again. Yield: 30 g.

### Example-15: Purification of 2,5-dichloro-3-(5-(3,4-dihydroxy-5-nitrophenyl)-1,2,4-oxadiazol-3-yl)-4,6-dimethylpyridine 1-oxide (Opicapone)

Dissolved the Opicapone (100 g) in the mixture of dimethyl sulfoxide (360 ml) and ethyl acetate (840 ml) at 60-65° C. and methanol (500 ml) was added and stirred at the same temperature for 60 minutes. Methanol (1900 ml) was added to reaction mass at 60-65° C. and stirred for 1 hour at the same temperature. Cooled the reaction mass to 25-30° C. and stirred for 60 minutes at the same temperature. Filter the solid, washed with mixture for methanol and water to get the title compound. Repeated same purification once again.

Yield: 88 g; PXRD pattern of the obtained compound is illustrated in FIG. 2, Particle size: D10: 1.882 μm; D50: 3.694 μm and D90: 6.759 μm; Purity by HPLC: 99.90%; Impurity content by HPLC: Desnitro N-oxide impurity: Not detected; Methoxy N-oxide impurity: Not detected; Des N-oxide impurity: 0.08% and Methoxy impurity: Not detected.

1. A process for the preparation of Opicapone of formula-(I) 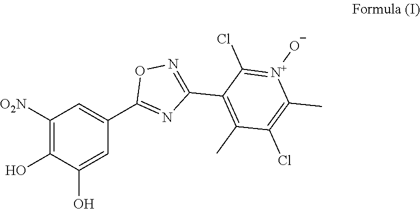 [SMILES: Cc1c(Cl)c(C)[n+]([O-])c(Cl)c1-c1noc(-c2cc(O)c(O)c([N+](=O)[O-])c2)n1]
comprises one or more of the following steps:
a) reacting the compound of formula (II) with compound of formula (IV) to provide the compound of formula (VII), 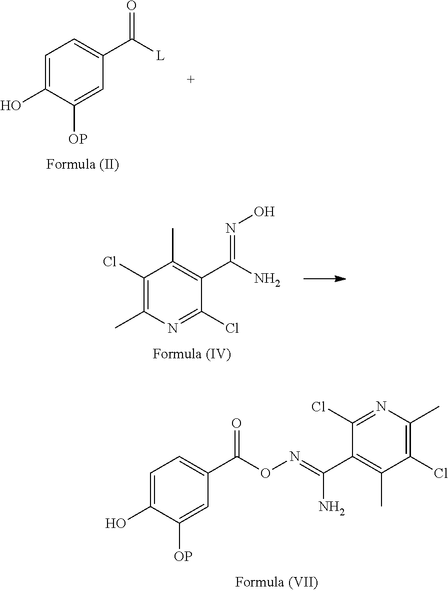
wherein, P refers to a protecting group such as methyl, ethyl, n-propyl, isopropyl, n-butyl, isobutyl, acetyl, trifluoro acetyl, benzyl, benzoyl and the like thereof;
L is —OH or any leaving group selected from halo, alkoxy, alkylsulfonyl or aryl sulfonyl,
b) cyclizing the compound of formula (VII) to provide compound of formula (V), 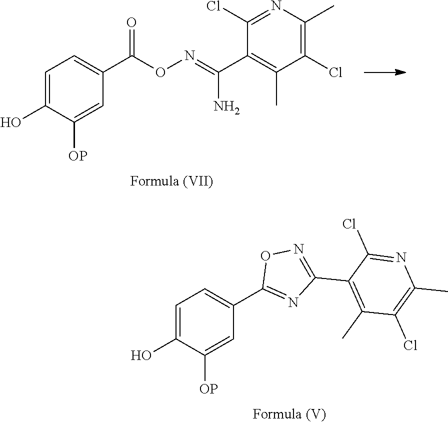
wherein, P is same as defined above;
c) nitrating the compound of formula (V) to provide compound of formula (VI) 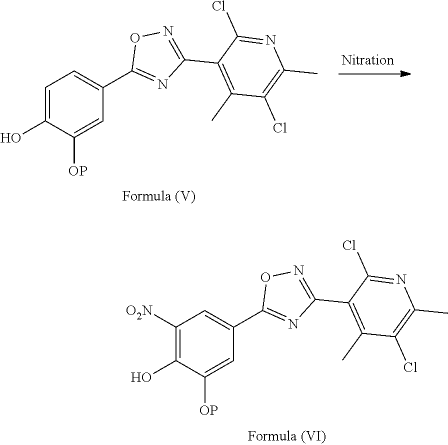
d) converting compound of formula (VI) to Opicapone of formula (I).

2. The process according to claim 1, wherein compound of formula (VII) in step-a) is isolated as a solid.

3. The process according to claim 1, wherein the compound of formula (V) is prepared without isolation of compound of formula (VII) as a solid.

4. The process according to claim 1, wherein the reaction in step-a) and b) is carried out in presence of base is refers to inorganic base or organic base; inorganic base selected from “alkali metal carbonates” such as sodium carbonate, potassium carbonate, lithium carbonate and the like; “alkali metal bicarbonates” such as sodium bicarbonate, potassium bicarbonate and the like; “alkali metal hydroxides” such as sodium hydroxide, potassium hydroxide, lithium hydroxide and the like; alkali metal hydrides such as sodium hydride, potassium hydride, lithium hydride and the like; alkali metal amides such as sodium amide, potassium amide, lithium amide and the like; ammonia such as liquor ammonia, ammonia gas, alcoholic ammonia and the like; and organic base selected from dimethylamine, diethylamine, diisopropyl amine, diisopropylethylamine, diisobutylamine, triethylamine, tertiary butyl amine, benzyl amine, “alkali metal alkoxides” such as sodium methoxide, sodium ethoxide, potassium methoxide, potassium ethoxide, sodium tert.butoxide, potassium tert.butoxide, lithium tert.butoxide and the like; pyridine, 4-dimethylaminopyridine (DMAP), N-methyl morpholine (NMM), 2,6-lutidine, lithium diisopropylamide; organosilicon bases such as lithium hexamethyldisilazide (LiHMDS), sodium hexamethyldisilazide (NaHMDS), potassium hexamethyldisilazide (KHMDS) or mixtures thereof.

5. The process according to claim 1, wherein when L is —OH, the compound of formula (II) in step-a) is activated by treating it with N,N-dicyclohexylcarbodiimide (DCC), N,N-diisopropylcarbodiimide (DIC), N-(3-dimethylaminopropyl)-N-ethylcarbodiimide (EDC) and N-(3-dimethylaminopropyl)-N-ethylcarbodiimide hydrochloride (EDC·HCI); carbodiimide is used in combination with an additive such as 1-hydroxy-7-azabenzotriazole (HOAt) or 1-hydroxy-1H-benzotriazole (HOBt); suitable Uranium reagent is selected from O-(7-azabenzotriazol-1-yl)-1,1,3,3-tetramethyluronium hexafluoro phosphate (HATU) and O-(benzotriazol-1-yl)-1,1,3,3-tetramethyluronium hexafluorophosphate (HBTU).

6. The process according to claim 1, wherein nitration in step-c) is carried out by using nitrating mixture (a mixture of nitric acid and sulfuric acid or a mixture of nitric acid and acetic acid), sodium nitrate, potassium nitrate, calcium nitrate, cupric nitrate and the like or mixtures thereof.

7. The process according to claim 1, wherein the compound of formula (VI) converted into Opicapone as per the process exemplified in the description.

8. A compound selected from:
i) a compound of formula (V) 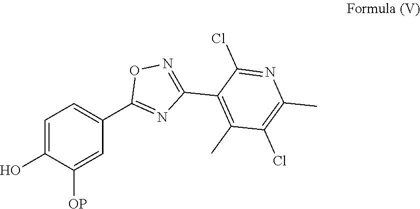
wherein P is methyl, ethyl, n-propyl, isopropyl, n-butyl, isobutyl, acetyl, trifluoro acetyl, benzyl, benzoyl and the like thereof
and
ii) a compound of formula (VII) 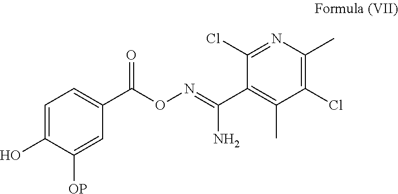
wherein P is methyl, ethyl, n-propyl, isopropyl, n-butyl, isobutyl, acetyl, trifluoro acetyl, benzyl, benzoyl and the like thereof.

9. (canceled)

10. The compound of formula (V) of claim 8, wherein P is methyl then compound of formula-(V) is (Va)

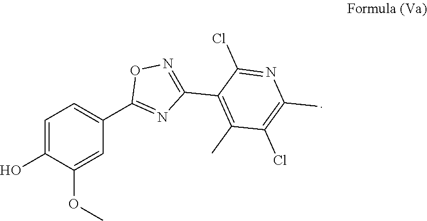 [SMILES: COc1cc(-c2nc(-c3c(Cl)nc(C)c(Cl)c3C)no2)ccc1O]

11. The compound of formula (VII) of claim 8, wherein P is methyl then compound of formula-(VII) is (VIIa)

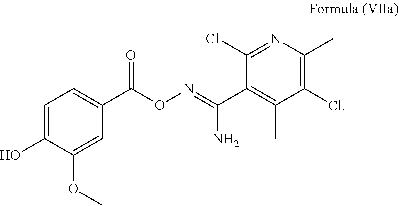 [SMILES: COc1cc(C(=O)O/N=C(\N)c2c(Cl)nc(C)c(Cl)c2C)ccc1O]

12. Converting the Compounds of formula (VII) and formula (V) of claim 8 to Opicapone of formula (I).

13. (canceled)

14. A process for the purification of Opicapone of formula (I), which comprises:
a) dissolving the Opicapone of formula (I) in the mixture of dimethyl sulfoxide and ethyl acetate,
b) combining an alcohol solvent or water with the solution obtained in step-a) and
c) isolating the Opicapone of formula (I).

15. The process according to claim 14, wherein dissolving of Opicapone of formula (I) in step-a) can be done at a temperature ranging from about 25° C. to reflux temperature of solvent or mixture of solvents used; alcohol solvent in step-b) is selected from methanol, ethanol, n-propanol, iso-propanol, n-butanol, iso-butanol, 2-butanol, tert-butanol, ethane-1,2-diol, propane-1,2-diol and mixtures thereof; isolating Opicapone in step-c) is by cooling the mixture to lower temperatures to precipitate the solid followed by filtration of the mixture.

16. Opicapone of formula-(I) obtained according to claim 14 is having purity >99% by HPLC.

17. (canceled)

18. (canceled)

19. Opicapone of formula (I) obtained according to claim 14 is having particle size distribution characterized by:
(i) D10 value is less than about 4 μm; or
(ii) D50 value is less than about 10 μm; or
(iii) D90 value is less than about 100 μm; or
(iv) a combination of (i), (ii) and/or (iii).

20. A pharmaceutical composition comprising, Opicapone of formula-(I) of claim 19 and a pharmaceutically acceptable carrier.
